(19) United States
(12) Patent Application Publication
(10) Pub. No.: US 2021/0027874 A1
(43) Pub. Date: Jan. 28, 2021

(54) COMPUTERISED METHOD FOR MANAGING END-TO-END PILL DISPENSING, PACKING, FORECASTING, ORDERING, AND SPACE PLANNING IN A NETWORKED SYSTEM

(71) Applicant: Miteshkumar Ishwarbhai Patel, Sacramento, CA (US)

(72) Inventor: Miteshkumar Ishwarbhai Patel, Sacramento, CA (US)

(21) Appl. No.: 16/591,049

(22) Filed: Oct. 2, 2019

Related U.S. Application Data
(63) Continuation of application No. 16/524,160, filed on Jul. 28, 2019.

(51) Int. Cl. G16H 20/13; G07F 17/00; G06F 16/2457; G07F 11/44; G07F 11/62; G16H 10/60; G07F 11/10

(52) U.S. Cl. CPC G16H 20/13; G07F 17/0092; G06F 16/24575; G07F 11/10; G07F 11/62; G16H 10/60; G07F 11/44

(57) ABSTRACT

The methods, automated and networked systems, and their components described herein are directed to applying logic flows and decision tree analysis, to manage pill dispensing and packaging of the dispensed pills into a blister pack in a high-volume pill dispensing environment. The described inventions provide end-to-end automation for a full cycle, start to finish, for the pill dispensing and packaging process.

The disclosed embodiments include several hardware components that are used to accomplish either partial or full pill dispensing and packaging process. Various determinations, logic and process navigations, real-time decision-making processes, and analysis of large amounts of data through high speed processors, are deployed through the provided process flow architecture described.

A computerized automated method for managing end-to-end medical pill dispensing and packaging process in a private computerized networked environment is described. The method also provides inventory management and space planning and optimization solutions. The method utilizes deep learning concepts to constantly improve the process and make it efficient and faster.

The method is used for dispensing and packaging medical pills in a blister pack for a patient in accordance with a prescription that is specific to the patient. The hardware used by the method is communicatively connected through a private computerized network. The hardware components include one or more computing devices comprising processor hardware, a plurality of laptop or mobile devices, one or more robot car parking stations, pack separators, drug dispensers, robot cars, manual verification stations, printer stations, sealing stations, unloading stations, and storage and delivery stations.

The method uses a central server that includes a processor, a system memory, and a decision logic module, wherein the processor is coupled to the system memory through an input/output (I/O) interface, and the decision logic module is communicatively connected to the processor.

The central server that includes a processor activates a prescription extraction flow module to extract a prescription from a client computer. These prescription include patient data and medication data. The patient data includes the patient's name and patient's date of birth, and the medication data includes a list of one or more medical pills prescribed for the patient, the quantity of pills for each of the one or more medical pills prescribed for the patient, and the hours of administration (HOA) of the one or more medical pills. It may include additional information such as addresses.

The extracted prescription are stored in the system memory of the central server and processed to separate the patient data from the medication data.

The decision logic module and a pack processing flow module communicate to develop a pack process flow plan. This plan details step by step sequential flow for dispensing and packaging the one or more medical pills prescribed for the patient into a blister pack. The robot car transports a blister pack from one station to another based on this pack process flow plan and as instructed by the processor.

At its outset, the mobile robot car retrieves an empty blister pack, wherein the mobile robot car includes a plurality of wheels and a receiver for receiving path guidance instructions from the processor.

The decision logic module selects a pill dispenser for populating the retrieved empty blister pack with the one or more medical pills prescribed for the patient. The selected pill dispenser is within the computerized networked environment and communicatively coupled to the central server through a private network.

The mobile robot car transports the retrieved empty blister pack to the selected pill dispenser where the blister pack is either completely or partially filled by the pill dispenser.

The processor sending the medication data from the stored prescription to the selected pill dispenser and instructs the pill dispenser to dispense pills into the empty blister pack in accordance with the hours of administration of the prescription. The pill dispenser then populates the blister pack accordingly.

The processor also formats the extracted prescription in a label printable format which is then printer by a selected printer. The blister pack is transported to the printer and the label is applied onto it.

The mobile robot car transports the populated blister pack from the printer to a sealing station where the label is sealed onto the blister pack.

The system executes a deep learning module to analyze the execution of the pack processing flow plan and revise the pack processing flow plan for its next execution. It does so by scoring the current path taken and matches it with simulated alternative paths to determine a path with the best score.

The system also provides optimal space planning solutions. It does so by receiving a plurality of requirements and factors, such as number of blister packs to be processed and typical volume of blister pack processing to be expected by the location. It also receives configurations such as dimensional layout of the space available to house the end-to-end medical pill dispensing, medical pill packaging, and medical pill inventory system. It further receives restrictions, such as blocked spaces where equipment cannot be placed, blocked zones where equipment cannot be placed, height restrictions, and temporary or permanent fixtures.

The system then analyses the requirements, factors, configurations, and restrictions to develop a layout for housing the end-to-end medical pill dispensing, medical pill packaging, and medical pill inventory system. It further evaluates and optimizes the developed layout by taking into account a logical and sequential flow of dispensing and packaging a blister pack and produces a final layout.

The system also performs inventory management and ordering of pills for a current as well as future cycles of prescriptions that are to be filled. The system provides a dashboard of ordering preferences and also provides a budgeting tool that can be tweaked by the user of the system to develop an optimized inventory budget and ordering plan.

### CONTINUATION INFORMATION

This application is a continuation-in-part of an application Ser. No. 16/524,160, titled “A NETWORKED AUTOMATED SYSTEM FOR MANAGING END-TO-END PILL DISPENSING, PACKING, AND SUPPLY-CHAIN.” having Miteshkumar Ishwarbhai Patel and Raj Kalpesh Patel as inventors, filed on Jul. 28, 2019. Application Ser. No. 16/524,160 is also incorporated herein by reference.

### FIELD OF THE INVENTION

The present invention relates to a method for managing end-to-end pill dispensing and packaging of a blister packet with medical pills in accordance to a prescription. Specifically, the present invention relates to an automated computerized method that uses a plurality of hardware devices that are networked together, and applies logic flows, deep learning, and automated decision tree analysis, to manage pill dispensing and packaging of the dispensed pills into a blister pack in a high-volume pill dispensing environment.

### DISCUSSION OF THE RELATED ART

Silo systems and individual components are used to dispense and fill blister packets with pills. The systems often do not have the capability of communicating with other components to perform a complete cycle of operation thereby requiring several operations and manual intervention to package a blister pack from start to finish.

In some instances, current systems use different components for achieving each step of the process, for example, a system may have partial process of dispensing performed by a machine while the remaining portion of the process, such as sealing the blister packet manually performed.

As such, an end to end system that is integrated, automated, and includes processes to communicate and manage the dispensing and packaging from start-to-end of the blister is needed.

### SUMMARY OF THE INVENTION

The methods, automated and networked systems, and their components described herein are directed to applying logic flows and decision tree analysis, to manage pill dispensing and packaging of the dispensed pills into a blister pack in a high-volume pill dispensing environment. The described inventions provide end-to-end automation for a full cycle, start to finish, for the pill dispensing and packaging process.

The disclosed embodiments include several hardware components that are used to accomplish either partial or full pill dispensing and packaging process. Various determinations, logic and process navigations, real-time decision-making processes, and analysis of large amounts of data through high speed processors, are deployed through the provided process flow architecture described.

A computerized automated method for managing end-to-end medical pill dispensing and packaging process in a private computerized networked environment is described. The method also provides inventory management and space planning and optimization solutions. The method utilizes deep learning concepts to constantly improve the process and make it efficient and faster.

The method is used for dispensing and packaging medical pills in a blister pack for a patient in accordance with a prescription that is specific to the patient. The hardware used by the method is communicatively connected through a private computerized network. The hardware components include one or more computing devices comprising processor hardware, a plurality of laptop or mobile devices, one or more robot car parking stations, pack separators, drug dispensers, robot cars, manual verification stations, printer stations, sealing stations, unloading stations, and storage and delivery stations.

The method is used for dispensing and packaging medical pills in a blister pack for a patient in accordance with a prescription that is specific to the patient. The hardware used by the method is communicatively connected through a private computerized network. The hardware components include one or more computing devices comprising processor hardware, a plurality of laptop or mobile devices, one or more robot car parking stations, pack separators, drug dispensers, robot cars, manual verification stations, printer stations, sealing stations, unloading stations, and storage and delivery stations.

The method uses a central server that includes a processor, a system memory, and a decision logic module, wherein the processor is coupled to the system memory through an input/output (I/O) interface, and the decision logic module is communicatively connected to the processor.

The central server that includes a processor activates a prescription extraction flow module to extract a prescription from a client computer. These prescriptions include patient data and medication data. The patient data includes the patient's name and patient's date of birth, and the medication data includes a list of one or more medical pills prescribed for the patient, the quantity of pills for each of the one or more medical pills prescribed for the patient, and the hours of administration (HOA) of the one or more medical pills. It may include additional information such as addresses.

The extracted prescription are stored in the system memory of the central server and processed to separate the patient data from the medication data.

The decision logic module and a pack processing flow module communicate to develop a pack process flow plan. This plan details step by step sequential flow for dispensing and packaging the one or more medical pills prescribed for the patient into a blister pack. The robot car transports a blister pack from one station to another based on this pack process flow plan and as instructed by the processor.

At its outset, the mobile robot car retrieves an empty blister pack, wherein the mobile robot car includes a plurality of wheels and a receiver for receiving path guidance instructions from the processor.

The decision logic module selects a pill dispenser for populating the retrieved empty blister pack with the one or more medical pills prescribed for the patient. The selected pill dispenser is within the computerized networked environment and communicatively coupled to the central server through a private network.

The mobile robot car transports the retrieved empty blister pack to the selected pill dispenser where the blister pack is either completely or partially filled by the pill dispenser.

The processor sending the medication data from the stored prescription to the selected pill dispenser and instructs the pill dispenser to dispense pills into the empty blister pack in accordance with the hours of administration of the prescription. The pill dispenser then populates the blister pack accordingly.

The processor also formats the extracted prescription in a label printable format which is then printer by a selected printer. The blister pack is transported to the printer and the label is applied onto it.

The mobile robot car transports the populated blister pack from the printer to a sealing station where the label is sealed onto the blister pack.

The system executes a deep learning module to analyze the execution of the pack processing flow plan and revise the pack processing flow plan for its next execution. It does so by scoring the current path taken and matches it with simulated alternative paths to determine a path with the best score.

The system also provides optimal space planning solutions. It does so by receiving a plurality of requirements and factors, such as number of blister packs to be processed and typical volume of blister pack processing to be expected by the location. It also receives configurations such as dimensional layout of the space available to house the end-to-end medical pill dispensing, medical pill packaging, and medical pill inventory system. It further receives restrictions, such as blocked spaces where equipment cannot be placed, blocked zones where equipment cannot be placed, height restrictions, and temporary or permanent fixtures.

The system then analyses the requirements, factors, configurations, and restrictions to develop a layout for housing the end-to-end medical pill dispensing, medical pill packaging, and medical pill inventory system. It further evaluates and optimizes the developed layout by taking into account a logical and sequential flow of dispensing and packaging a blister pack and produces a final layout.

The system also performs inventory management and ordering of pills for a current as well as future cycles of prescriptions that are to be filled. The system provides a dashboard of ordering preferences and also provides a cost management tools that can be tweaked and optimized by the user. The system, using its analysis and software tools, analysis the user preferences and provides a final result, such as ordering the pills based on the factors tweaked by the user.

### BRIEF DESCRIPTION OF THE DRAWINGS

The accompanying drawings are included to provide further understanding of the invention and constitute a part of the specification. The drawings listed below illustrate embodiments of the invention and, together with the description, serve to explain the principles of the invention, as disclosed by the claims and their equivalents.

FIG. 1 illustrates a networked automated system for end-to-end pill dispensing, packaging, and delivery according to the disclosed embodiments;

FIG. 2 illustrates an architectural diagram for communication between the hardware components of the networked automated system according to the disclosed embodiments;

FIG. 3 is a block diagram that illustrates the process of retrieving a prescription and using the retrieved information for dispensing and packing its associated pills according to the disclosed embodiments;

FIG. 4 is a flowchart that describes the steps of extracting the prescription data according to the disclosed embodiments;

FIG. 5 illustrates a block diagram for deep learning according to the disclosed embodiments.

FIG. 6 illustrates a flowchart for scheduling the dispensing of pills from a pill dispenser according to the disclosed embodiments;

FIG. 7 illustrates a flowchart for utilizing one or more pill dispensers for either partially or completely filling the prescription according to the disclosed embodiments;

FIG. 8 illustrates a flowchart for determining inventory and ordering pills according to the disclosed embodiments;

FIG. 9 illustrates a flowchart for forecasting inventory and suggesting supply chain alternatives according to the disclosed embodiments; and

FIG. 10 illustrates a block diagram depicting the process involved in developing an optimized layout to house the end-to-end pill dispensing, packaging, and delivery system according to the disclosed embodiments.

### DETAILED DESCRIPTION OF THE PREFERRED EMBODIMENTS

Aspects of the invention are disclosed in the accompanying description. Alternative embodiments of the present invention and their equivalents are described without parting from the spirit or scope of the present invention. It should be noted that like elements disclosed below are indicated by like reference numbers in the drawings.

The methods, automated and networked systems, and their components described herein are directed to applying logic flows and decision tree analysis, to manage pill dispensing and packaging of the dispensed pills into a blister pack in a high-volume pill dispensing environment. The described inventions provide end-to-end automation for a full cycle, start to finish, for the pill dispensing and packaging process.

The disclosed embodiments include several hardware components that are used to accomplish either partial or full pill dispensing and packaging process. Various determinations, logic and process navigations, real-time decision-making processes, and analysis of large amounts of data through high speed processors, are deployed through the provided process flow architecture described.

The described systems are modular and stackable that provide for scaling up or down depending on the volume of pill packing desired. The modularity and stickability is also utilized for adjusting to various physical space constraints at the location of the system.

The system also has functionality to perform forecasting and ordering of pills as needed. When inventory is running low, whether its for the current build or a future build, the system alerts the user and provides projections on when the inventory will run out and what is the cost of building up inventory. It also allows the user options to order pills or does so automatically.

The system also provides space layout optimization for pharmacies, clinics, medical facilities or other locations that house the system. It determines several factors, constraints, and provides a layout which is then optimized and customized based on the dimensions of the layout space and all provided variables.

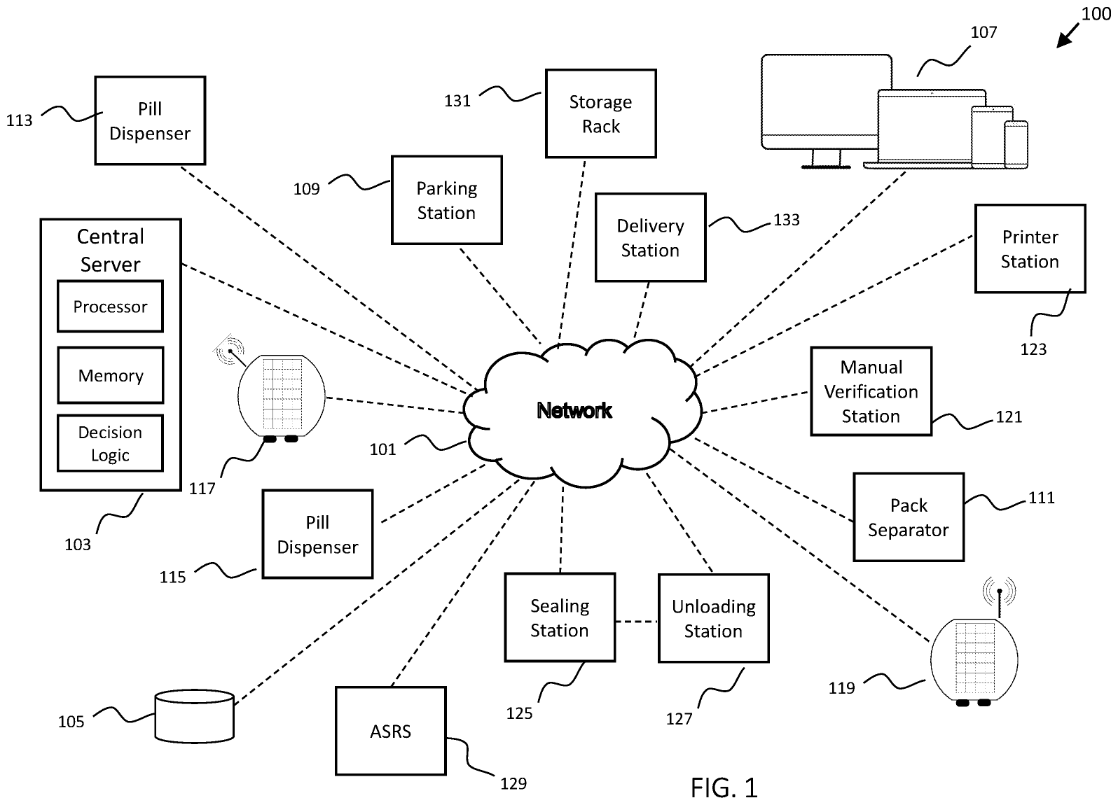

FIG. 1 illustrates a networked automated system for end-to-end pill inventory management, dispensing, packaging, and delivery according to the disclosed embodiments.

The system of FIG. 1 provides a representative environment in which the invention can be implemented. Aspects of the invention described in FIG. 1 includes hardware components as well as some general communications and process instructions or computer-executable instructions, such as routines executed by a general-purpose data processing device, such as, a server computer, laptop or personal desktop computer, and/or a mobile device. Those skilled in the relevant art will appreciate that the invention can be practiced with other communications, data processing, communications, or computer system configurations, including: wireless mobile devices, Internet capable devices, and wearable computers, multi-processor or micro-processor-based systems or programmable consumer electronics and the like. The terms “computer,” “server,” are used interchangeably and may refer to any of the above devices and systems.

Further, although certain functions are described as being performed exclusively on a single hardware device or in a single process, the invention can also be practiced in distributed environments where functions or modules are shared among disparate processing devices and various changes are made to the single process, such as running parallel processes, or multi-process to accomplish a similar result as that described in the invention. Further, if a distributed computing environment is used, various program modules may be located in both local and remote memory storage devices.

System 100 may be located in a pharmacy, hospital, a manufacturing warehouse, a clinic, or other medical services facility. The network of devices depicted is able to include any number of devices and any various devices including, but not limited to, a central server 103 connected to the network 101, a source 105, a plurality of laptop or mobile devices 107, a robot car parking station 109, a pack separator 111, plurality of drug dispensers 113-115, a plurality of robot cars 117-119, a manual verification station 121, a printer station 123, a sealing station 125, an unloading station 127, an ASRS station 129, a storage rack 131, and a delivery station 133.

As mentioned earlier, not all the components are required in all the operations and the components are interchangeable, modular, and stackable to fit both the space and volume requirements for pill dispensing and packaging.

The central server 103 includes a processor, memory, and decision logic. The one or more processors assists in processing information and instructions for operation of the system 100. The processor, or microprocessor, is coupled to memory, which can be non-transitory storage medium, for executing program instructions. The processor is also configured to provide instructions to various components of the system and those instructions result execution of the operational instructions results in coordination between the components of the system to perform automatic and autonomous end-to-end pill dispensing and packing.

One example of the processor's operation is to obtain patient prescription data from the source 105. Then storing this data into the memory of the central server. Also obtaining the inventory of pills to fulfil the prescription from a plurality of pill dispensing machines. Further obtaining availability of one or more robot cars for transporting a blister package to a desired pill dispensing machine. Storing all the data obtained into the memory. Communicatively working with decision logic to analyze the stored data and process instructions for a particular robot car 117 to travel to a particular dispensing machine for filling the prescription. In performing it tasks, the processor also interacts and works with decision logic on an as needed basis. This is just one example of the processor's function; the example has been simplified to provide a glimpse into its operations. Several other operations and functionality that is described in the figures below is also executed by the processor.

The central server connects to all the components of the system, i.e., the source device 105, the plurality of laptop or mobile devices 107, the robot car parking station 109, the pack separator 111, plurality of drug dispensers 113-115, the plurality of robot cars 117-119, the manual verification station 121, the printer station 123, the sealing station 125, the unloading station 127, the ASRS station 129, the storage rack 131, and the delivery station 133, and uses its processor, memory, and decision logic to analyze and perform operations on the entire system thereby acting as a central command. The central server's database includes a list of patient information, prescription information, medication information, and other information needed to dispense and package pills into a blister pack, as well as component details, such as capability, availability, scheduling etc. Alternatively, the central server 103 obtains some of the unavailable data through the source 105. As mentioned earlier, the connectivity with the components allows the central server 103 to provide instructions to all the components such that they can communicate with each other and operate in accordance with the optimal plan of operation.

The decision logic is part of the central server 103. Decision logic or decision logic device or means is responsive to the processor, also part of the central server 103, and is used to automatically engage in a decision-making process. Once data on operation of the system, including operation, availability, and inventory of each component in the system is accessed and stored into the memory, the decision logic takes over to find the best path, the most efficient process, or the best utilization of each component and provides the decision logic for the processor to process and execute commands resulting in the actual functionality and operation of the system.

In one example, the decision logic may determine the number of cars parked in a parking station and decide to send an unmarked robot car to a separate parking station if it is efficient to navigate to the separate parking station in a shorter amount of time. It may also access traffic data stored in the memory to suggest the best path for the robot car 117. Path determination, system operational efficiency, supply chain management, component utilization is just a few of the processes that are analyzed by the decision logic, several other processes, some of which are described in further detail below are also contemplated.

To accomplish its decision, such as a decision involving path determination, system operational efficiency, supply chain management, component utilization etc., the decision logic means compares the data obtained by the processor and stored in the memory with certain threshold values or a counter. The threshold values and counter may be pre-determined or the decision logic may constantly update the threshold or the counter based on historical data and deep learning to refine the model and suggest a new threshold or counter. These thresholds or counters can be used to determine path determination, system operational efficiency, supply chain management, component utilization.

For example, a threshold may be to allow a maximum of 3 robot cars on a particular path or to a certain parking station, if the new robot car enters the system, and the threshold is reached, the decision logic may provide alternative path towards parking stations. The decision logic may also keep track of the time periods during which each component is available and operational as well as its capacity, for example, to determine utilization, underutilization or over utilization.

The decision logic applies decision tree analysis and deep learning over time to make decisions that improve the efficiency and performance of the system. The deep learning aspect is performed over time where each system cycle is stored and analyzed for its performance. The subsequent cycle is improved based on system performance, hurdles, time delays, and any system issues observed in the previous performance and used to enhance future cycles. With each cycles of data and each new problem encountered, the system continues to learn and improve subsequent cycles.

For example, the deep learning module evaluates the total amount of time taken to perform each step of the pill dispensing and packaging process. Its also analyses delays and downtime in performing each step of the pill dispensing and packaging process. It further analyses distance travelled by the robot car in performing each step of the pill dispensing and packaging process. It also scores the results of the path taken, total time, delays and downtime, and distance travelled.

The deep learning module also runs simulations and develops alternative paths that can be used by the robot car to run through one cycle of pill dispensing and pill packaging. The deep learning module scores these alternatives paths and revises the process flow plan continuously by selecting the path that has the highest score.

The system includes connectivity to a plurality of client devices 107, such as laptops, desktops, tablets, or other mobile devices. These devices 107 may include at least one other client application that is configured to receive information and content, such as patient information, medication information, or dispensing and packaging data, from another computing device or from the central server 103.

The client application may include a capability to provide and receive textual content, or multimedia information, such as patient information, medication information, or dispensing and packaging data. The client application may further provide information that identifies itself, including a type, capability, name, or the similar identifying information. They may also uniquely identify themselves using phone number, mobile identification number (MIN), an electronic serial number (ESN), mobile device identifier, network address, such as IP (Internet Protocol) address, media access control (MAC) layer identifier.

The client computing devices 107 may also be configured to communicate a message, such as through email, short message service (SMS), or multimedia message service (MMS), to the central server or another computing device.

One or more of the devices is able to be an end user, a company and/or another entity. In some embodiments, peer-to-peer sourcing is implemented. For example, the source of the data to be compared with is not on a localized source but is found on peer sources.

As mentioned earlier, system 100 also includes a source 105. The central server 103 is communicatively coupled to the source through the network 101. The source or source device 105 can be any device that contains a source such as a searchable database, web pages, medical records, EMR (electronic medical records), patient prescription data, physician notes, patient medical history, or any other information or device that provides information relating to patient, patient's medicines, prescriptions, medication side effects, or dispensing, filling, and packaging of medications. The source device is also capable of accessing the Internet.

The network 101 can be any network or number of networks such as the Internet, cloud networks, an intranet, Ethernet, a LAN/WAN, either wired or wireless, satellite, a combination of any of the mentioned networks that allow communications.

The pill dispenser 115, also referred to as pill dispensing robot, drug dispenser, or pill dispensing machine includes various compartments or storage locations for storing pills and customized medications. These pill storage areas can be a canister, a manual fill tray, or other storage boxes. The canister is a storage box with a rotating mechanism that dispenses a pill upon rotation of the internal mechanism. Several drawers, in varied sizes and shapes, both large and small can store a plurality of canisters in each drawer, for example one drawer can store 16 canisters. Several storage areas, canister drawers, and plurality canisters are located in the pill dispenser 155 and together they can store over 256 varieties of pills and medications. Additional pills are also stored in manual fill trays.

The dispensing section of the pill dispenser includes a hopper, which is a funnel shaped conduit through which pills are dropped into a tray. The tray, also known as pre-fill tray or collection tray, have a grid of slots that mimic the grid of slots in a blister pack. The grid of slots represents the days of the week and the times of the day. Once pills are collecting in the tray, after they have been dropped through the hopper, the collected pills are then transferred to a blister pack. The blister pack is located in the robot car, such as robot car 117 or 119, that has been assigned to deliver the empty blister pack to the dispensing machine and collect the filled blister pack for transporting it to another station in the system 100.

The pill dispenser 115 may also include other components such as packing plate, which acts as a second collection plate after the pre-fill tray, an additional hopper, which may be smaller than the hopper, and other components that allow dispensing and collection of pills to be ultimately transferred to a blister pack. The dispensing robot also has its own processor and memory. The processor communicates with Central Server 103 through network 101, Source 105 through network 101 or through Central Sever 103. The dispensing machine through its processor obtains the prescription of a particular patient and saves it in its memory. It uses this patient prescription data to dispense pills from selected canisters, manual fill trays, and canister drawers that correspond with the pills for the prescription. The processor intercuts the appropriate canister, manual fill tray, or other storage areas in the pill dispenser 115 that hold the corresponding pill to be dispensed and dropped into the hopper to be delivered to the pre-fill tray and ultimately in the appropriate slot into the blister pack. Since the blister pack slots correspond with days of the week and times of the day, each pill is designated for a specific slot such that the pill is to be taken on a certain day and certain time of the day that corresponds with the hours of administration (HOA) that is in the patient prescription.

In another embodiment, the pill dispenser receives all the dispensing commands and instructions from a mobile application or directly from the central server 115. It then dispenses the pills as instructed for a specific patient and populates the slots of the blister pack. Other methods of instructing the pill dispenser 115 are also contemplated.

Although details of one individual pill/drug dispenser has been described, the network is not so limited and includes a plurality of pill dispensers. The number of pill dispensers in a system 100 depends on several factors. These include volume needs, space requirements, churn rate to pack a certain number of blister packages per day, the type of facility, e.g., large hospital or pharmacy to a smaller facility or clinic or nursing home. The number may also depend on the amount and variety of medication that needs to be stored and dispensed. For example, a large hospital that has a high patient turnover rate and a large number of patients in their facility may requires a faster and higher volume of blister packs. If the hospital has the space, since it has a high requirement to package a large amount of pill packs, it is likely that multiple pill dispenser will be part the system 100. Likewise, a smaller facility, such as a smaller clinic or nursing home or pharmacy that has lesser requirements and a lower capacity to fill a lower number of blister packs will likely have just 1 or lesser pill dispenser.

The robot car parking station 109 is a modular station where multiple robot cars 117-119 can be parked. It is essentially an electro mechanical structure that is used for parking robot cars. The parking station can have several floors and one robot car 117 can fit on each floor. The parking station may also be designed to accommodate more than one car on each floor. The number of floors can vary and the parking station is modular such that floors can be added or removed as desired or required by the system 100. In yet another example, there will be a robot car parked on a single floor.

The number of robot car parking slots, parking floors, and number of robot cars parked on each floor also depends on the total number of robot cars used by system 100 as well as the need to address a certain volume of pill dispensing. For example, a large system that has a high volume, high capacity, and a high requirement to package a large amount of pill packs and includes several pill dispensing machines 115 will likely have a higher number of robot cars 117-119 and likely a higher number of robot car parking stations, which results in a larger number of robot car parking slots within a station as opposed to a smaller system that has lesser requirements and a lower capacity to fill a lower number of blister packs.

The parking station may be a transitory stop for the robot car 117-119 or it may be housed and parked there for a longer term when it is not being used. The system 100 through its central server 103 keeps track of all robot cars and where they are parked at any given time. As such, the system 100 is aware whether to send a robot car to a particular parking station based on the parking station capacity, reservation, or availability.

A pack separator 111 is a device that includes a mechanism that separates one blister pack from another. Since blister packs are stacked on top of each other it becomes necessary to separate and obtain a single blister pack for filling the medications for a single patient. The separator functions to separate a single pack and then have it loaded onto the robot car 117 for transporting it to its next destination. To do so, the pack separator uses a plurality of sensors. These sensors are capable of finding the separation lines between the stacked blister packs. A couple alternatives to separate the packs include the use of either a fork mechanism or a feed screw. Once separated, the blister pack is dropped into the robot car.

The robot car 117, also shown at 119, and also referred to as robot transport or dose car or robot vehicle transport, is a mobile car that can transport a blister pack from one location to another location. It is guided and directed by the central server 103. It may also be guided and directed by other means, such a decision logic of the central server 103, or another database or mobile application that can track the movement of components in the system 100.

The robot car 117 travels in tracks. The tracks allow movement in a straight path. It also includes several stop points. The stop points are where the robot car 117 switches its XY direction. The robot cars 117 can also be lifted to a higher level and transferred from one track to another.

The robot car 117 includes wheels and certain electronics that allow it to navigate from one location to another. It also includes either an antenna, transmitter, or other type of communication module that can receive and send signals and receive instructions that guide its path of travel.

The manual verification station 121 is a mechanical structure designed for the manual intervention. This station may or may not be used depending on its need. In one embodiment, the manual verification station is used as an error correction step in the pill dispensing and packing process. It can also be used as a secondary check or verification of the pills already populated into a blister pack.

In one possible use, it is required when an error is detected by the pill dispenser 115 during its dispensing process. The error may be that a wrong pill has been populated in the wrong slot of the blister pack or that pill from an unintended canister was dropped that does not match the prescription for the patient.

The manual verification station is communicatively connected to the pill dispenser 115 through network 101. It is also communicatively connected to the central server 103 through network 101. Aside from the errors mentioned above, other errors that are detected in dispensing or filling that are either detected by the pill dispenser 115 or reported to the central server 103, such as for example, missing pills from a certain cell of the blister pack, can also be flagged for inspection and then transported to the manual verification station 121. A particular blister pack that is flagged is transported to the manual verification station either manually or by the robot car 117. At this station, each cell of the blister pack is verified and checked in accordance with the patient's prescription to ensure that all the pills are correctly populated in the appropriate time slots for the patient.

The printer station 123 is a mechanical structure that is used for printing labels that are placed on the blister packs. The printer station is communicatively connected to the pill dispenser 115 through network 101. It is also communicatively connected to the central server 103 through network 101.

The labels printed by the printer station include patient data and prescription data. The patient data can be patient name, address, phone number or other patient related information. The pill data can be hours of administration, medication information, side effects of the medication, and other information that may be helpful to the patient in administering the medication. It can also include details for taking the medication before or after a meal.

Although a single printer station 123 is disclosed in system 100, the system may include several printer stations that are situated in various locations. In one embodiment, the printer station is physically located next to or very near a pill dispenser. This is to facilitate the printing of the labels and placing them on the blister pack as a next step after the blister pack has been filled by the pill dispenser. In operation, a robot car 117 may carry the filled blister pack from the pill dispenser 115 to the printer station 115 upon receiving instruction from the central server 103 or its decision logic module.

The number of printer stations 123 in a system depend on the amount of printing and labeling needed, i.e. the amount of blister packs to be filled. It also depends on the need to address a certain volume of pill dispensing, i.e., a large system such as a hospital system that has a high volume, high capacity, and a high requirement to package a large amount of pill packs and includes several pill dispensing machines 115 will likely have a higher number of printer stations as compared to a smaller system that has lesser requirements and a lower capacity to fill a lower number of blister packs will have just one or fewer printer stations.

The sealing station 125 is a mechanical structure designed for sealing the label that was previously printed by the printer station 123. To do so the sealing station applies a calculated amount of heat on the label and the heat causes the label to stick to the blister pack. Since the label contains glue at its bottom, upon application of the heat, the glue melts or liquefies and bods the label with the blister pack thereby resulting in the labels sticking to the blister pack.

Although a sealing station 125 is disclosed in system 100, the system may include several sealing stations. As mentioned above, this is dependent on the need, volume, and churn rate or blister packs to be prepared in a day or within a certain time frame. As such larger facilities, such as hospitals, that has a high volume, high capacity, and a high requirement to package a large amount of pill packs will also include a higher number of sealing stations in their system 100.

In operation, the sealing step comes after the label is printed by the printer station. As such, it makes logical flow to have the sealing station located nearby to the printing station. However, the sealing station can be situated in various locations.

In operation, a robot car 117 may carry the filled blister pack from the pill dispenser 115 to the printer station 115. Once a label has been placed on the blister pack, the robot car 117 may then proceed to the sealing station to have the label sealed.

Since the sealing station is also communicatively connected to the pill dispenser 115, central server 103, and printer station 123 through network 101, it can receive commands from the central server 115 that can keep it apprised of the process of the packaging and when to expect the robot car 117 to arrive at the sealing station.

The unloading station 127 is a station where a completed blister pack gets unloaded from the robot car 117. The unloading station is a two-axis system consists of a vertical axis and a horizontal axis. It uses a pick-and-drop mechanism and to pick up the blister pack from the robot car 117 and place it for storage or next step. In operation, the robot car 117 transports a completed pill pack that has all the pills required for a particular patient along with a heat-sealed label to the unloading station. The pill or blister pack which sits on or within the robot car 117 is then picked up at this unloading station.

System 100 may include one or more unloading stations 127. They may be located in various locations or near a delivery station. The number of unloading stations, as explained with other components, depends on the amount of volume, churn rate, and type of facility. Typically, a facility may have just one unloading station, however, large warehousing or distribution operations may include more.

The unloading station 127 is communicatively connected to central server 103 through network 101. It sends data back to the central server 103 that allows the central server 103 to keep track of the number of packaged units or blister packs completed and delivered or shipped. This helps in inventory management and also to access the amount of completion and the amount left to be completed for the day or for the time duration.

The ASRS station 129 includes a conveyor and a gripper. It is basically a mechanism for loading storage racks and storing blister packs into the storage racks. The ASRS station can be an independent mechanism or it can be connected communicatively through the network to the central server 103 and also to the other components in the system 100. The system may include multiple ASRS stations.

Once the blister pack is unloaded at the unloading station 127, it is then placed in storage at the storage rack 131. The storage rack 131 may include a plurality of moving mechanisms that assist in placing the completed blister pack in storage. It may also include several floors and layers that allow the blister pack to be stored at various locations. It also includes location tracking through RFID means that is used by central server to track the location of the storage rack. Similar to other components, there may be one or more storage rack that are connected through the network 100 to the central server 103.

The storage rack is a wheeled device that can be transported from one location to another by maneuver it on its wheels. In one embodiment, the storage rack has several rack levels, where each rack level has 8 slots or 8 storage positions. For example, the storage rack, along with its storage slots together may allow the storage of 96 blister packs. Alternatively, it could be more or less depending on the need and the structure of the parking station.

The delivery station 133 may be optional. In some instances, the final blister packs loaded onto the storage racks may be the end of the process and in other instances a delivery station 133 may be included. The delivery station 133 basically monitors all the blister packages that are being sent out of the facility or send out of the dispensing and packaging station to their intended recipients. It can also be used as a final check step to ensure that pill packages have been properly packaged and have the corresponding label on them.

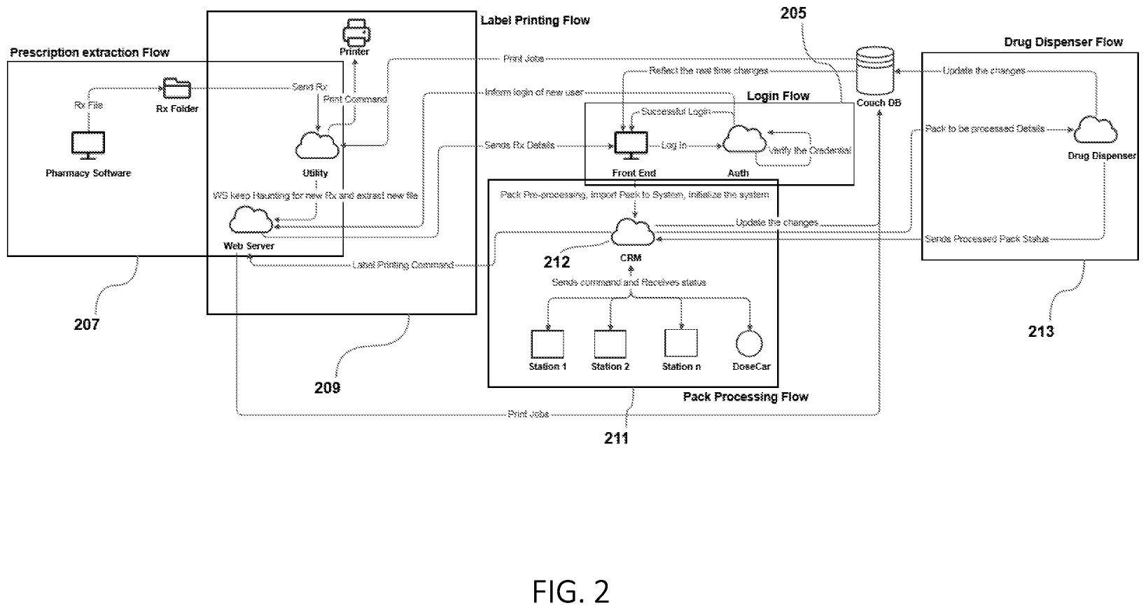

FIG. 2 illustrates an architectural diagram for communication between the hardware components of the networked automated system according to the disclosed embodiments. As described earlier, system 100 includes a plurality of hardware components that function to dispense pill and package them into a blister pack. System 100 may have one or more of each type of hardware components as needed to fulfill the volume and speed requirements of a particular facility. There may also be a combination of different number of types of hardware components where one type may be more than another. For example, the system 100 may include one of each or two or more of pill dispensers 115, but only one of unloading station 127, it depends on the needs and can be customized.

The hardware components can be categorized by those that dispense and package, those that transport, those that provide pre- and post-dispensing support, such as storage, or those that provide commands for operations.

As far as the physical dispensing and packing goes, the system 100 uses plurality of drug dispensers 113-115, a manual verification station 121, a printer station 123, a sealing station 125, an unloading station 127, an ASRS station 129, which includes a storage rack 131 and a gripper.

The pre- and post-dispensing hardware components include pack separator 111 and storage racks 131.

The transportation between these components/stations is performed by a plurality of robot cars 117-119. These use parking facilities, also referred to as parking stations 109 that are networked and in the system 100.

System 100 also includes several operations modules that perform specific functions. These modules include login flow module 205, prescription extraction flow module 207, label printing flow module 209, pack processing flow module 211, and drug dispenser flow module 213. In one embodiment, the hardware components mentioned above are also grouped based on these operational modules.

Each module provides operation support, processes data, and directs components, including hardware components, within its control to perform certain functions to accomplish a task, such as dispensing pill or printing a label for a blister pack.

System 100 include a login flow module 205. This module 205 is communicatively connected to the Couch DB, label printing flow module, and pack processing flow module 211.

The key role of this login flow module 205 is to provide a successful login, verify credentials, verify and authorize access, and act as a front end for all login and access related applications. Once the access is granted, the login flow module 205 also determines the level of access depending on their user. For example, a third-party application that is to send prescription data may not be allowed internal access and just sufficient access to provide the data based on patient identification whereas a manager of the system who ensures operations is likely to get full access. The access may also be for internal systems that need to monitor inventory and order supplies and pills—for such systems, an access to pill inventory may be granted. The level and type of access depends on the need and authorization and can be changed by the login flow module 205.

The prescription extraction flow module 207 works with a computer, such as a server computer, laptop computer, or the like that has a database for storing patient information. The prescription extraction flow module 207 executes by compiling or accessing medical records, also referred to as “Rx records” for a patient.

The medical records may be made available subject to HIPAA rules and regulations, as well as federal and state law, or any laws and regulations governing the use of medical records and privacy.

These records may be located at the pharmacy or across databases for large pharmacies. As depicted in this Figure, the records are located in a Pharmacy that uses a type of Pharmacy Software to parse and store the patient data in a database. Alternatively, the records may be stores at any other facility, such as a doctor's office, hospital, nursing home or centrally on the cloud or on an EMR system.

To access these records, the prescription extraction flow module 207 would need authorization, and in some instances require login information or other type of verification for it to be allowed access to databases that house the patient data. Along with the system 100 and module 207, upon authorization, the patient may also have access to the data from the pharmacy, prescriber, or central prescription service.

In operation, the data is stored in the pharmacy or at a physician's office or a central EMR system. The data includes all of patient's medical records, prescriptions, basic contact information, medical history, and other information related to a patient's medical background and prescriptions.

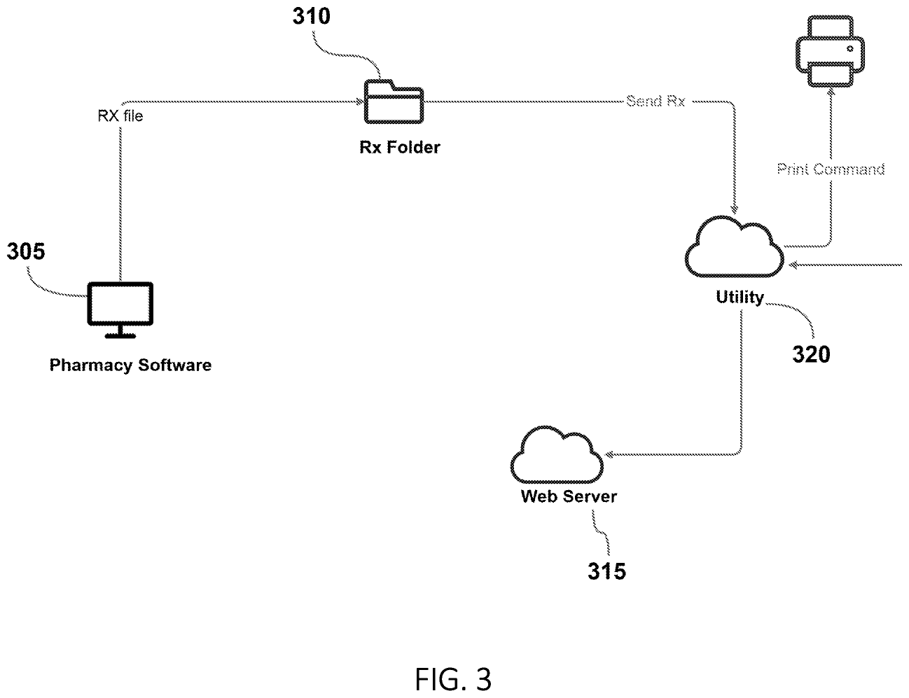

The prescriptions include details of pills, hours of administration (HOA) of the pills and any recommendations from the physician, such as to take the pill after a meal. The details of extracting and using this prescription information are provided in FIG. 3 below.

FIG. 3 is a block diagram that illustrates the components involved in retrieving a prescription and using the retrieved information for dispensing and packing its associated pills according to the disclosed embodiments.

The components involved include Pharmacy Software 305, RX folder 310, Utility Server 320, Web Server 315, and Printer 123. The Pharmacy Software 305 is an algorithm that collects the patient prescription data, parses through the data, and stores it in a local database or in a local memory of the computer on which the Pharmacy Software 305 is housed.

The RX folder 310 is a folder that is created by system 100 and it stores the prescription date in the central server. The RX folder 310 may also be located on another computer that is connected to the prescription extraction flow module 207.

The utility server 320 communicates with the central server of the system 103 to exchange data and allow the central server to perform operations and analysis of the prescription data obtained.

The Web Server 315 is also another outlet to the Internet where a web-based application or access to cloud or other online storage can be accessed. In this embodiment the prescription data would be accessible online or through the cloud.

The printer 123, as explained above is a mechanical structure that is used for printing labels that include details of pill intake and are placed on the blister packs. The printer station is communicatively connected to the central server 103 as well as to the utility and web servers 315 and 320.

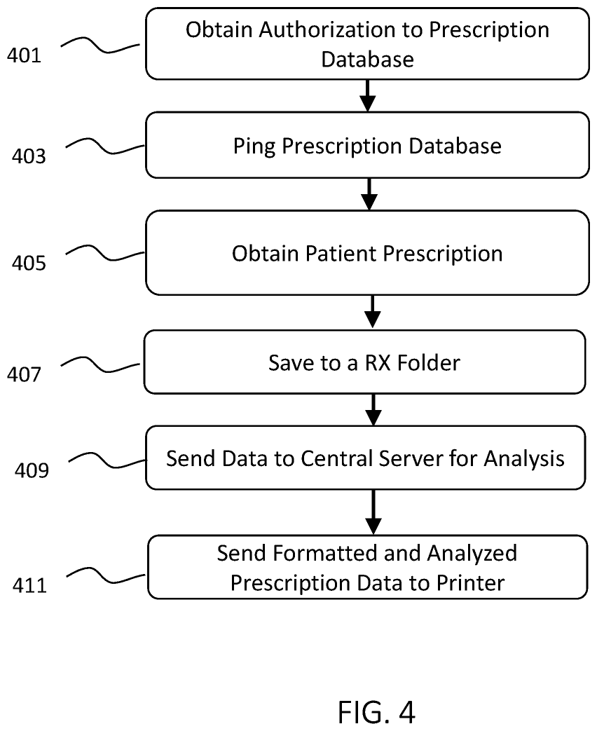

FIG. 4 is a flowchart that describes the steps of extracting the prescription data according to the disclosed embodiments. Although a particular process is described in a sequence of steps, these steps can be modified in sequence, combined, and also skipped depending on the database from which the prescription data is being retrieved. For example, authentication and access login may not be needed of the data housed is internal to the system 100 and within the HIPAA complaint data sharing platform.

At Step 401, authorization with the Pharmacy Database that contains the Pharmacy Software 305 is obtained. This authorization may be through a login or a 2-step verification system that provides a code and asks the system to enter the code to get access to the patient data. Other means of authentication, such as Multi-factor authentication or Two-factor authentication is also contemplated.

The multifactor authentication consists of a method that grants access to the computer asking for information only after successfully presenting two or more pieces of evidence. This could include the patient's date of birth, their driver license numbers, medical record number, etc. The information is something the user and only the user knows and can verify the user's identity.

Likewise, Two-factor authentication, which is related to multi-factor authentication, user identity by using a combination of two different factors such as information they have or some information they should know about their medical history.

The access may also be granted once HIPAA compliance is determined. If the computer or the system is not HIPAA compliant, then it may need to incorporate HIPAA standards to be able to access this information, in brief, this means the standard for sensitive patient data protection should be applied. Systems must have physical, network, and process security measures in place and follow them to ensure patient data is protected and not shared outside the authorized circle.

If other systems, people, or contractors will have access to the data, such as subcontractors and any other related business associates must also be in compliance. The HIPAA Privacy Rule establishes national standards to protect patient medical records and other personal health information and applies to health plans, as well as to their health-related information such as prescriptions that are to be retrieved by the system 100.

Once authorization is obtained the access is granted to obtain patient related data. As such, at Step 403, the prescription database is pinged or queried to obtain patient's prescription.

A query is essentially a request for patient data from a table in the database that is associated with the patient. Once queried with a patient ID or some other form of patient identifier, data is generated as results returned by Structured Query Language (SQL) or other forms. SQL Client may also be used which is a JAVA-based database tool used for administration and it allows the viewing of database structure and use of SQL commands.

In this instance, since the goal is to dispense and package a blister pack for a patient, the query is defined specifically to ask for medications, such as pills, that are prescribed for that specific patient such that they may be dispensed and filled according to the prescription. Further, information relating to the medications, such as an hour of administration, any intake instructions, side effects or the like may also be queried.

At Step 405, the prescription database responds with prescriptions and details of the prescription, such as name and type of pills, quantity of pills, intake regimen, hours of administration etc.

At Step 407, the obtained information is saved to an intake folder on System 100. The intake folder is also referred to as Rx folder or prescription folder. In one embodiment, this folder is saved in the memory of the central server 103. Alternatively, it may be received by the central server 103 and then sent to any of the pill dispensers, such as 113 or 115, whoever is assigned by the central server 103 to dispense the medication, such that they prescription can be used and adhered to while dispensing.

The Rx folder may also be shared and stored with the utility server that is part of the prescription extraction flow module 207.

At Step 409, once the prescription is saved to an Rx folder, it is sent to the central server 103 for processing. As mentioned earlier, the Rx folder can be saved in multiple locations such as in the memory of the central server 103, memory of the pill dispenser that will be fulfilling the prescription, the utility server, or some other location in the system 100. Regardless of its storage location, the central server 103 is sent the details of the Rx folder.

The central server 103 receives this data and analyses the prescription. It does so by parsing the data into multiple segments that can be used for filling the blister packs as well as printing a label that is to be placed on the blister pack.

In one embodiment, the processor in the central server 103 extracts all the pill data and correlates that data against the current inventory in the system. It does so to determine which pill dispenser 113 or 115 has all or partially all of the pills that are associated with the prescription. It does so for multiple reasons, for example for scheduling the appropriate pill dispenser, for determining inventory or lack thereof for fulfilling the prescription, and to queue the prescription and schedule hardware resources in the system 100 for its fulfilment. Once the analysis is complete, a pill dispenser is identified and the prescription is either sent to the pill dispenser or specific commands are sent that direct the pill dispenser on which pills to dispense from which canister/storage location within the pill dispenser.

In another embodiment, the system suggests specific canisters that house the needed pills. It does so for all the pill dispensing robots in the system. System also suggests replenishing pills when it detects that the inventory of robot is not sufficient for any type of pill. This proactive approach and advance planning makes the system run at a higher efficiency without having to stop and wait until a canister is empty of pills to then fill it after the fact.

The processor in the central server 103 also extracts data for printing purposes. Since it is a requirement, and if it is not, a preferred best practice, to place a label on the blister pack that contains the prescribed medications, the processor extracts information that it parses, formats, and sends to a printer in a format that is to be printed on the label.

For example, in the United States, the legal requirements for a label that goes on the blister pack is set by federal law and state statutes. It is as such required that the prescription label may be the only source of instructions on how to take their medicines and so it needs to be complete. Several other details, such as safety, and side effects may also be required. Additional requirements vary and may include serial number of prescriptions, date of prescription or date of its filling, name of prescriber or doctor, Name of patient, patient's date of birth, the patient's address, and directions for taking medication, including precautions, if contained in prescription. The processor extracts this information from the prescription it obtains from the prescription database and formats it to ensure all the requirements are fulfilled and placed in an acceptable and readable format.

At Step 411, the central server 103, through its processor send the data that is to be printed to a designated printer in the system 100. The printer is also queried for its availability and queued in line with the pill dispensing schedule.

Referring back to FIG. 2, label printing flow module 209, performs functions and steps that result in the printing of a label that is placed on top of the blister pack. The components utilized by the label printing flow module 209 include one or more printers 123 in the system 100, the utility server, and the web server.

In one embodiment, the label printing flow module 209 moderates, schedules, and communicates with other modules and the central server to print a label that is to be placed on the blister pack. The label printing flow module 209 through its web server receives a label printing command. The command may be sent through the central server or an authorized user. The command may also be in response to the label printing flow module 209 pinging another server or authorized user.

The label printing flow module 209 through its web server receives prescription data, also as mentioned earlier, Rx data, that includes details on contents that are to be printed on the label for the blister pack. The web server is connected to the Internet and able to connects across networks to databases that hold the prescription data and is able to extract or receive that data once authorization is confirmed.

After receiving the prescription data, which may already be sent in a format that is ready to be printed, the web server sends it to the utility server. Alternatively, the web server may have capabilities to convert the data into a printable format for the label.

The utility server's WS (Web Server) continues to ping or haunt the web server for new Rx data or Rx file. Once it receives it from the web server, it extracts the relevant data that is to be printed onto the label for the blister pack. As mentioned earlier, in one embodiment, the Rx printable data may already be sent in a format that is ready to be printed by either the authorized user or the central server.

If the data is sent already formatted by the central sever, it is due to the processor of the central server 103 interacting with the decision logic of the central server 103 to decide what is required by local and federal laws, relevant and pertinent information that is useful for intake of medication, and instructed by the physician and extract that information to be formatted and sent to relevant servers and printers to be printed.

When the Utility server receives the data and processes the data, it then sends the data to a local printer 123. It does so by determine that the printer 123 is functional, available, and able to perform the print job. Alternatively, the functionality, availability is predetermined by the central server.

The printer 123 receives the print job and prints the label that is to be affixed to the blister pack. The label includes written text in one side and a heat sensitive glue on the other side. The heat sensitive glue can be loosened or melted when heat is applied to affix the label onto the blister pack.

The pack processing flow module 211 works with drug dispenser module and a plurality of stations 1-n and the dose car to pre-process and perform all functions needed prior to actual dispensing of the pills into the blister pack by a pill dispenser. The pack processing module 211 includes a Central Resource Manager (CRM) 212 that acts as the brain, i.e. receives data, processes, data, and guides and directs functions.

These functions include, but are not limited to, pre-processing retrieval of the blister pack from a stored location using the pack separator, scheduling resources, such as the transportation resource, such as the dose car, to pick up and deliver the blister pack from the pack separator to the intended pill dispenser, organizing data that may be needed by the pill dispenser to dispense the pills, checking system availability for both functionality, inventory, and availability of pill dispensers to determine which pill dispenser can be used to fulfill the prescription. Alternatively, some of the functions may be performed by the central server through its processor and the decision logic. There may also be a combination of certain decision that have been made prior by central server and some shared between the central server and the pack processing flow module 211.

The pack processing flow module's CRM 212 may also check on the performance of a pill dispenser or the status of fulfillment either mid-cycle through the fulfillment and dispensing process or after. It may also ping the pill dispenser on a routine predetermined interval to determine its current status, performance, and percentage fulfillment of the blister pack and process those updates and send them to the central server.

The pack processing flow module's CRM 212 also keeps track of number of blister packs processed by each pill dispenser in the system 100 and checks on inventory of pills in each pill dispenser. When the inventory starts to get low, of falls below a certain threshold, it may also inform the central server for additional pills to be added. It may also directly order the pills or provide an alert to the systems or resources that are responsible for filling the pills back above the threshold. Alternatively, it may just inform the central server and the central server may overtake the next necessary steps to ensure proper inventory is maintained and ordered in time to avoid any delay and disruption to the pill dispensing process.

The pack processing flow module's CRM 212 can send commands to any of the pill dispensing stations, such as Station 1-n, as well as to any and dose car to perform certain functions. For example, the pack processing flow module 211 may send a command to the dose car to fetch an empty pill pack from a storage area or to transport the pill pack from one station to another or to transport it to a certain pill dispenser along a certain path.

The pack processing flow module's CRM 212 may also send commands to the pill dispenser. These may include scheduling commands or to dispense in a particular order to start dispensing and then pause until certain pills are filled.

System 100 also includes a drug dispenser flow module 213. This module communicates with the Couch DB (database) and pack processing flow module 211. The drug dispenser flow module 213 communicates status of the pill pack dispensing, updates changes to its availability and functionality, receives pack processing details from other servers, and provides instructions to the pill dispenser to dispense pills.

In the event there are any disruptions, delays, or lack of inventory, the drug dispenser flow module 213 may communicate such details as part of their status update. It also sends data on the number of pill packs processed per day, per certain cycle, or in a defined time frame.

The status data, as well as any updates, inventory details, or availability and functionality detail it provides are stored in the Couch DB. The Couch DB data can be retrieved by the front end or the central server any time it desires an update or on a periodic time interval. The central server may obtain such data so that it keeps a check on the entire system 100 to ensure high performance, efficiency, and that pill packs are being packaged in an orderly and scheduled manner.

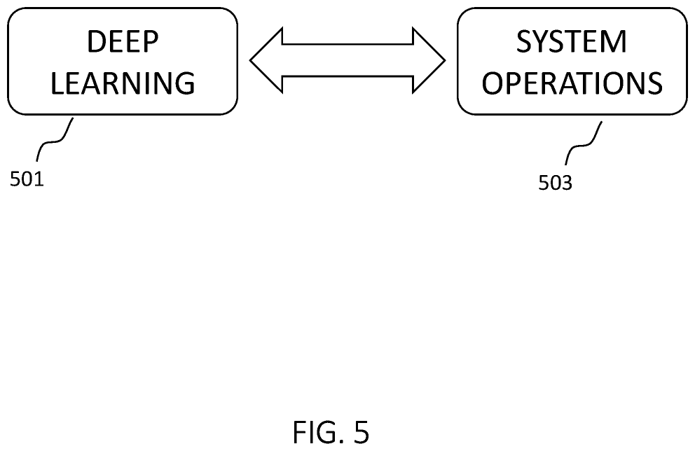

FIG. 5 illustrates a block diagram for deep learning according to the disclosed embodiments. The blocks represent a deep learning module 501 and a Systems Operations Module 503. The systems operations module stores all the operations performed by the system. These operations can be one cycle from start to finish of filling an empty blister pack and storing it or one cycle of operation for performing a space layout or one cycle of operation for preforming forecasting and budgeting. Several other operations and functionality of the system in addition to dispensing, packaging, ordering, forecasting, and layout planning have been described through the application and the system operations module stores all such operations. The operations are a set of instructions that are based on decision analysis, algorithms, and couched in software code and instructions. The deep learning module 501 receives the data from the systems operations module 503 and performs analysis to determine if any process could have been done faster, more efficiently, or with better use of resources. It also determines the performance of each function, performance of each hardware device, along with any errors, downtime, under performance, underutilization etc. The deep learning module provides suggestion on improvement for future cycles based on past performance. It also has the capability to develop new model and provide suggestions on which model or path would have resulted in a better outcome.

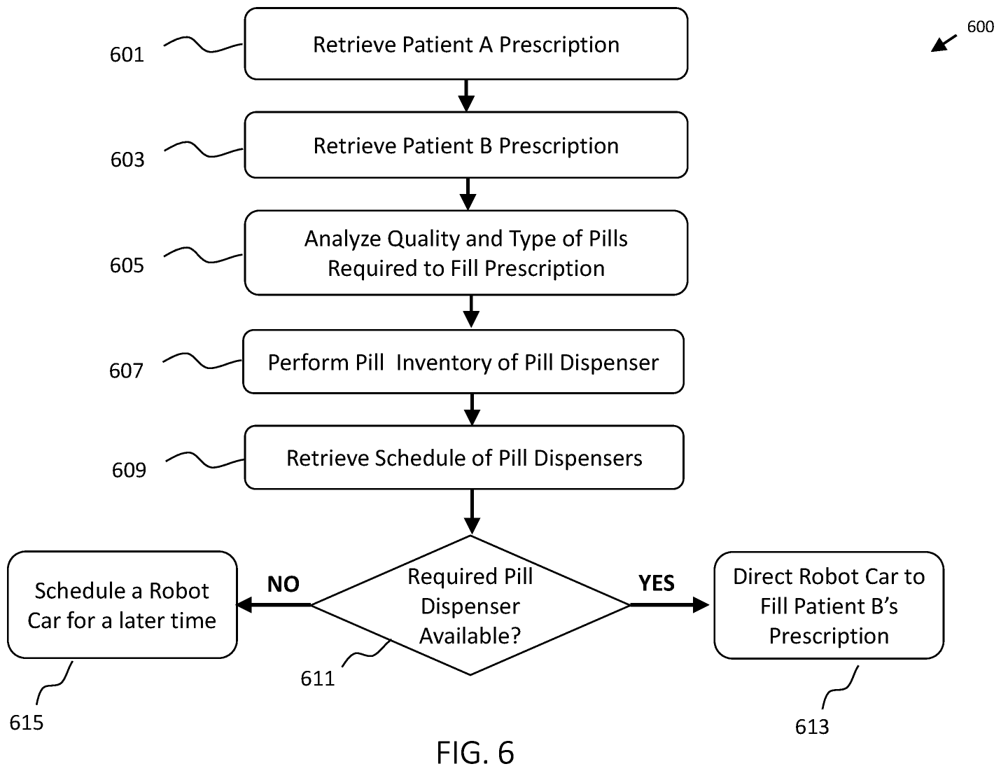

FIG. 6 illustrates a flowchart for scheduling the dispensing of pills from a pill dispenser according to the disclosed embodiments. The process exhibited in flowchart 600 determines the inventory of the pill dispensers required to fill all the pills in prescriptions A and B and based on the assessed inventory sends the robot car with the empty blister pack to the designated pill dispenser that contains the pills and is available for dispensing.

At Step 601, the system retrieves prescription A that is associated with a specific patient. Prescription A includes all the medications and pills that are required by the patient. Prescription A contains the names and quantities of each pill that is required to complete the prescription. The system retrieves prescription A from a database that houses such information. The database can be internal or external to system 100. If external, the system 100 obtains and complies with appropriate authorizations and HIPAA regulations to retrieve it from the database.

At Step 603, the system retrieves prescription B that is an associated with a specific patient. In a similar process as that used for retrieving Prescription A, the system retrieves prescription B that includes names and quantities of each pill that is required to complete the prescription. The system retrieves prescription B from a database that houses such information.

Although just two prescriptions, Prescription A and Prescription B have been described, the process can be used for any number of prescriptions that are to be filled in a particular time frame, such as for 1-day or one cycle of operation, or for a 24-hour period or some other defined time frame.

At Step 605, the system analyses the quantity and type of pills required to fill Prescription A and Prescription B. In so doing, the system 100 analyses the prescription and compartmentalizes various components of the prescription to identify each pill and its quantity required for dispensing and packing that prescription into a blister pack.

At Step 607, the system 100 performs an inventory of the pill dispenser in system 100. For example, if system 10 includes three pill dispensers, then system 100 determines inventory of pills in each of the three pill dispensers. If system 100 had recently performed an inventory of the pill dispensers, it keeps the log od the inventory and updates them as required. In another instance, the system 100 runs a routine check or a check prior to start pf a day or start of a new cycle to determine inventory. Keeping a tally also minimizes time utilized in running a pill inventory every time a new prescription comes in and instead the system, uses the updates and stored pill dispenser inventory information and updates it without having to run a query.

At Step 609, the system 100 retrieves the schedule of each pill dispenser in system 100. The schedule provides details of availability, priority, backlog, and timing of all the prescriptions that are being filled by each pill dispensers.

At Step 611, the system 100 determines which pill dispenser in the system is available to receive the new prescription and fill it. For example, it is possible that one of the pill dispensers may be running a current pill dispensing job to fill a particular prescription and may to be available to tale on the new prescription until it completes the filling of its current prescription. In another example all the pill dispenser s may be busy filling a prescription, however, one of them may be ready after the current fill while others may have 3 or 4 prescription fill jobs in queue.

At Step 613, upon a determination that a particular pill dispenser is available and contains the required pill in inventory to fill the prescription the system 100 sends the robot car to the identified pill dispenser for getting the pill filled in the blister pack. As shown in this example, the system 100 may also make changes such as send a robot car to get prescription B fill first before prescription A if the available pill dispenser contains inventory to fill the pills for prescription B but not prescription A. Other alternatives and similar changes may also be made to accommodate filling and prioritizing and re-prioritizing of dispensing to maximize the efficiency of the system and fill all prescriptions in the fastest time possible.

At Step 615, the system 100 may schedule the robot car for a certain time if all the pill dispensers are busy such that the robot car arrives at a designated pill dispenser when it is available to perform the dispensing.

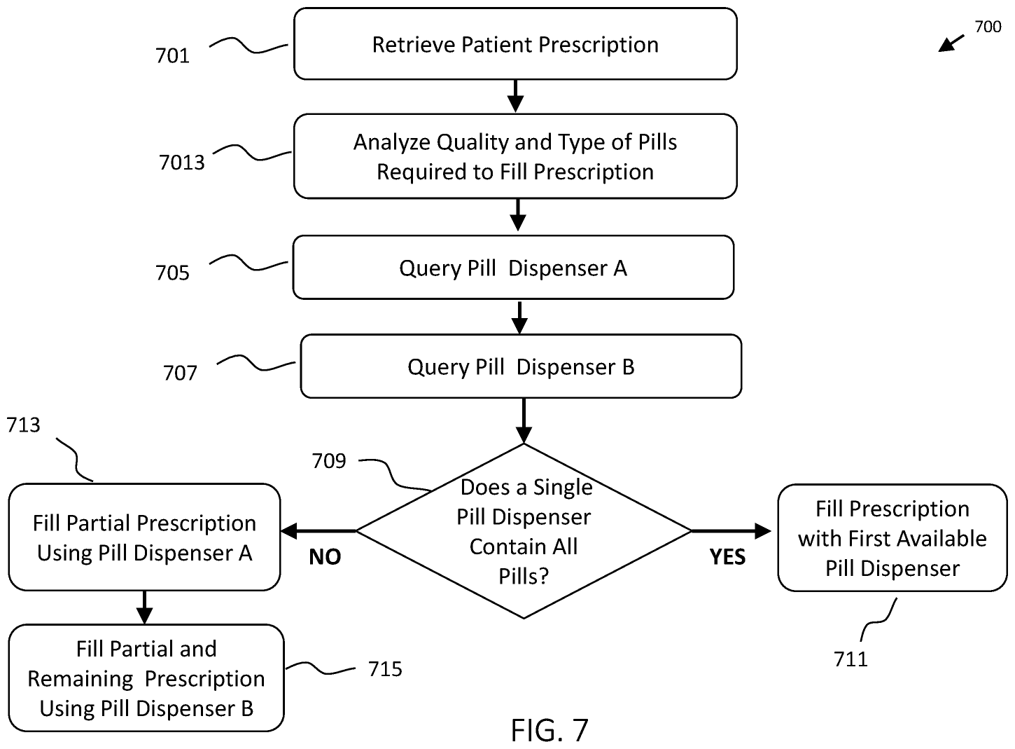

FIG. 7 illustrates a flowchart for utilizing one or more pill dispensers for either partially or completely filling the prescription according to the disclosed embodiments. The process exhibited in flowchart 700 determines the inventory of the pill dispensers in the system and determines if the dispensing and filling of a single blister pack needs to be split between two pill dispensers.

At Step 701, the system retrieves a single prescription. This prescription is associated with a specific patient. The prescription includes details, such medication type and quantity of pills that are required by the patient. The system retrieves prescription from a database, similar to that described in FIG. 6.

At Step 703, the system analyses the quantity and type of pills required to fill the retrieved prescription. It also determines the quantity of each pill required for the prescription.

At Step 705 and 707, the system 100 performs an inventory check of the pill dispenser A and pill dispenser B. For exemplary purposes, this embodiment has been limited to two dispensers in the system 100, however, the invention is not so limited and can accommodate anywhere from 1 to N number of pill dispensers.

The system 100 then determines inventory of pills in pill dispenser A and Pill dispenser B. It keeps a tally of the inventory. In another embodiment, it may run a routine check or a check prior to start of a day or the start of a new cycle to determine inventory. Keeping a tally also minimizes time utilized in running a pill inventory every time a new prescription comes in and instead the system, uses the updates and stored pill dispenser inventory information and updates it without having to run a query.

At Step 709, the system 100 determines if either pill dispenser A or pill dispenser B contains all the types of pills and the quantity required to fill the single prescription. If only one the pill dispensers, i.e. from dispenser A or pill dispenser B, has all the types and quantity of pills for the prescription, then at Step 711, the system 100 sends the robot car to the pill dispenser which can perform the complete fill.

In one embodiment, dispenser A may have some of the pills and pill dispenser B may have the remaining set of the pills. In such cases, the system 100 at steps 713 and 715 decides to have dispenser A and pill dispenser B split the dispensing of the prescription and together fill the blister pack one by one. As such, the robot car will first proceed to either dispenser A or pill dispenser B and get its partial fill and then proceed to the other dispenser for remaining fill. The system may also decide to send the robot car first to either dispenser A or pill dispenser B depending on who can perform the higher percentage of fill.

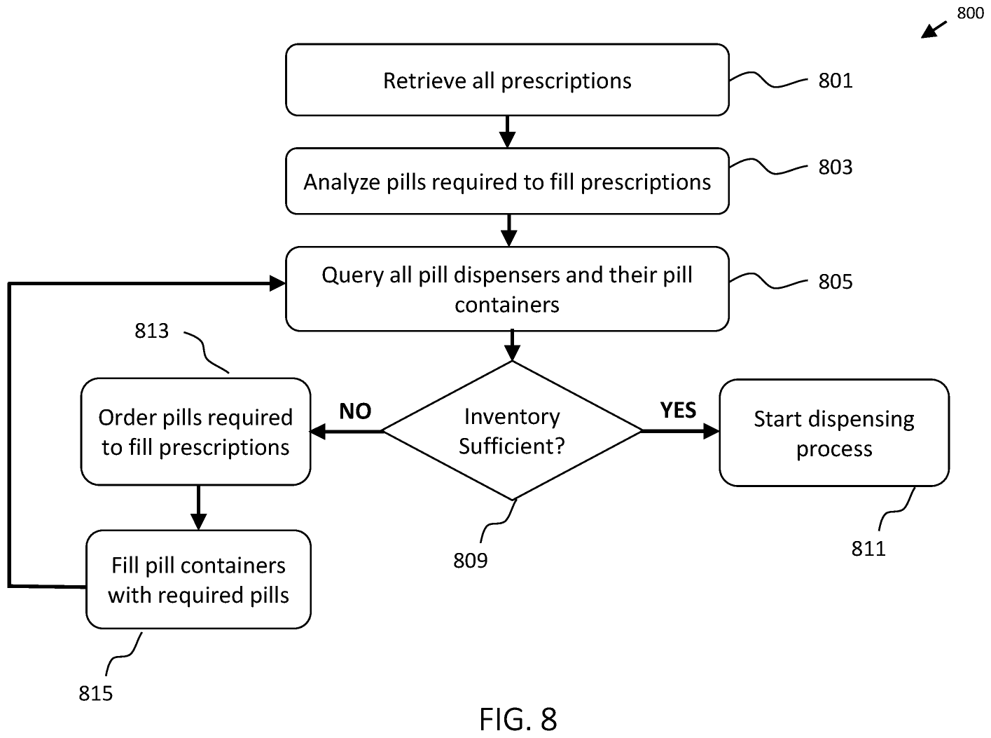

FIG. 8 illustrates a flowchart for determining inventory and ordering pills according to the disclosed embodiments. The process exhibited in flowchart 800 performs supply chain, inventory management, and ordering of pills. It is a management tool used by the system 100 to allow the system 100 to run efficiently and determine supply and demand based on number of prescriptions to fill in a specified amount of time.

At Step 801, the system retrieves all the prescriptions that are to be filled in a specified amount of time. Each of these prescriptions include details, such as type and quantity of pills that are required to fill each prescription. The system retrieves prescription from a database, similar to that described in FIG. 6.

At Step 803, the system analyses the quantity and type of pills required to fill the retrieved prescription. It also determines the quantity of each pill required for the prescription.

At Step 805 the system 100 performs an inventory of all the pill dispenser in the system 100. This includes an inventory of the containers, both manual containers and canisters that are placed in a plurality of drawers.

The system 100 then maps the total number of prescriptions to be filled in the specified time with the current inventory in all of the pill dispensers in the system. Alternatively, the system maps each single prescription with a pill dispenser to ensure that it is capable of filling the complete prescription. Alternatively, the system 100 allows partial filling as an option and takes that into account when calculating the inventory as a whole and not by each prescription to a pill dispenser.

At step 809, if a determination is made that the current inventory is sufficient to fill all the prescription in the specified time, then at Step 811, the system starts the dispensing and filling process.

However, if at step 809, a determination is made that the current inventory is not sufficient to fill all the prescription in the specified time, then at step 813, the system orders the required number of pills to fill the prescription.

At step 815, the ordered pills are filled in the containers of the pill dispensers. At this point the system 100 performs a feedback loop to Step 805 to recalculate the amount of inventory based on the number of prescriptions to be filled. The process may be repeated after each cycle of dispensing to keep a tab of inventory such that pills can be ordered in time for a future dispensing cycle.

The system may also look at future cycles of dispensing while it is currently performing dispensing for an earlier cycle such that it can order to allow replenishing of pills without having a delay.

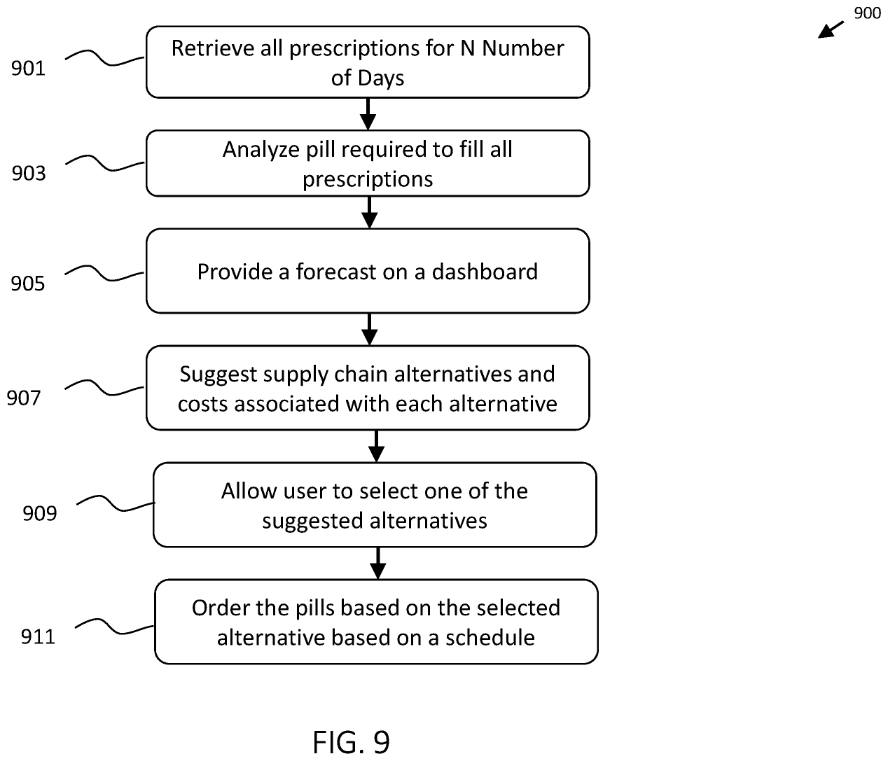

FIG. 9 illustrates a flowchart for forecasting inventory and suggesting supply chain alternatives according to the disclosed embodiments. The process exhibited in flowchart 900 perform an analysis to forecast future inventory needed to fulfil upcoming orders. The principles applied in providing forecast include deep learning and predictive analysis. The goal of the forecasting process is to retain adequate inventory to fulfil the pill dispensing demand while at the same time not build up too much inventory that would result in a higher cost. The forecasting process results in providing a display on a dashboard of its user, such as through a mobile application on a phone or on their display screen on their tablet or laptop. The displayed results may include options for the user to choose based on alternatives provided to build up their inventory while viewing the cost of such inventory. User may be provided additional options to chose or the user my customize and build their own solution based on the suggestions provided by the system.

At Step 901, the system retrieves all the prescriptions that are to be filled for N number of days in the future. Each of these prescriptions include details of type and quantity of pills that are required to fill each prescription. The system retrieves prescription from a database, similar to that described in FIG. 6.

At Step 903, the system analyses the quantity and type of pills required to fill the retrieved prescription. It may categorize the pills into groups, such as X number of Tylenol needed for next 10 days based on all the prescriptions that require Tylenol. It may further categorize based on amount of dosage required. Other variations of categorization are also contemplated.

At Step 905 the system 100 performs an analysis to and produces a forecast of inventory required to fulfill all the prescriptions for N days. The system 100 may provide this forecast on a day by day, week by week, or by any set time frame. The system may also provide granularity on specific pills that are needed. The system may also include date relating to a typical timeline required to order a specific pill and take than into account when providing forecasting suggestions. In some cases, a drug may have a lead time of 1-3 days while in other cases, such as with rare or orphan drugs, it may take weeks.

The forecasting is also based on deep learning and historical analysis. For example, as the system undergoes several cycles it develops a better understanding of usage of pills based on historical data. The more historical data it archives, the better it become in terms of predictive analysis and forecasting.

At Step 907, the system 100 provides alternatives and costs associated with each alternative. The results are displayed on the user's display screen or dashboard, such as table, mobile phone, laptop, computer, screen attached to the dispensing robot etc. The alternatives may range by cost, risk, and/or a gauge of comfort level. For example, a user may desire to take less risk have a higher comfort level that the pills are in inventory and such a user may build up a larger inventory for its own comfort. The system would allow the user to select such alternative and provide a cost of building up the excess inventory.

At Step 909, the system, by undergoing decision analysis provides a set of instructions, such as software instructions, that allow the user to select any of the alternatives provided and refine it to their own liking. The system, using the analyses and instructions provided presents on a plurality of options on a dashboard of a display screen. These options may also include several buttons or sliding scale pointers than can be maneuvered by the user from left to right, or top to bottom, thereby allowing the user to tweak several buttons and pointers to get a desired solution. For example, the user may adjust cost, risk, lead time, comfort level on the sliding scale to determine an optimal inventory build up that is customized for them and within their budget.

At Step 911, once a suggested solution is selected, or the user chooses to refine the solutions by adjusting the parameters, button, and sliding scale factors, the system 100 allows the user to order pills based on the inventory plan and schedule selected. The ordering may also be tiered based on dates such that all the inventory is not build up at one time but tiered over time to match the dispensing schedule or stay an X number of days ahead of the dispensing schedule.

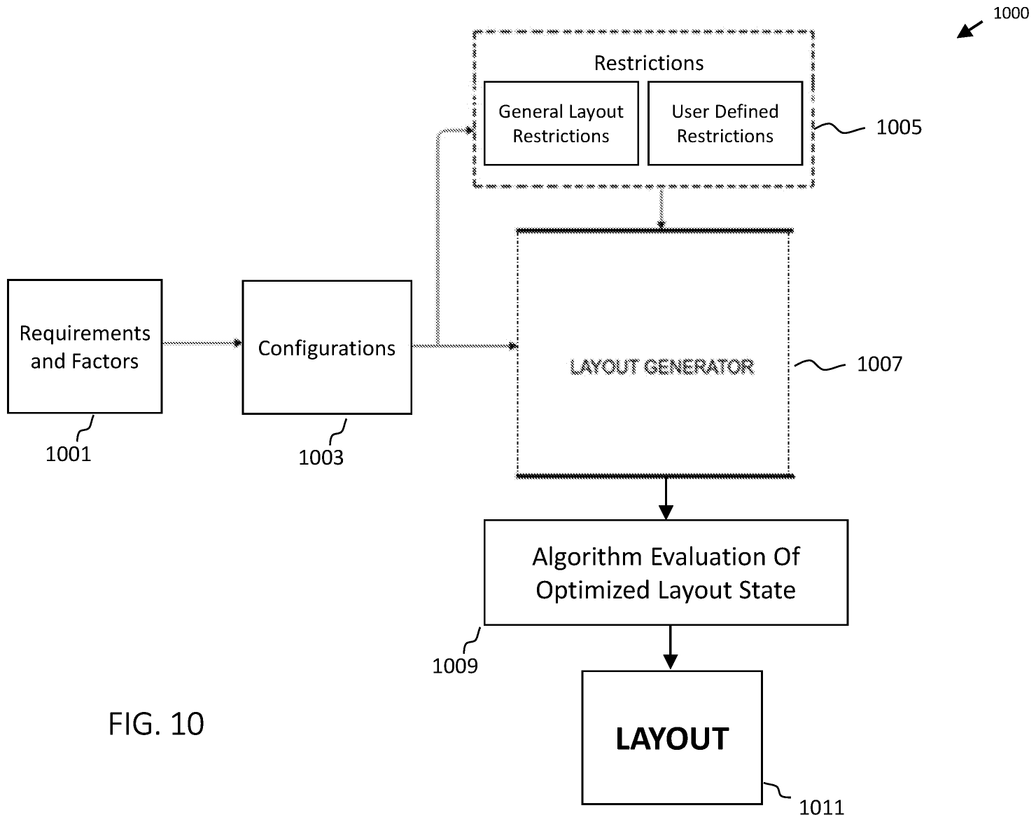

FIG. 10 illustrates a block diagram depicting the process involved in developing an optimized layout to house the end-to-end pill dispensing, packaging, and delivery system according to the disclosed embodiments.

The layout optimization is performed by the central server 103 to design and reconfigure the end-to-end pill dispensing, packaging, and delivery system such that is fits in the provided space and performs the required functions efficiently.

A requirements and factors module 1001 is provided. The module 1001 obtains a set of user requirements and factors and then provides them to the central server. These user requirements and factors may include quantity of blister packs that are to be processed per day, the type and nature of operations to be performed, and the budget and expenses involved in performing those operations.

The requirements may also include dimension of space as well as a layout. It may include vertical height of open space as well as any obstructions. It may also detail of other fixed and movable structures that located in the space where the end-to-end pill dispensing, packaging, and delivery system is to be located.

For example, a pharmacy that is required to dispense and package a high volume of blister packs will need a larger amount of space and resources compared to a pharmacy that has lesser volume requirements. However, if the pharmacy does not have floor space to house all the equipment needed, then, based on the requirements and space details provided, layout optimization may be reconfigured such that the end-to-end pill dispensing, packaging, and delivery system may be stacked vertically or use certain components that perform the required tasks while fitting within the allotted space, it all depends.

The requirements and factors 1001 are then used by the configurations module 1003 to determine the station configuration and the floor area. The station configuration is the use of stations such as pill dispenser, printing station, sealing station, storage station. The goal is to fit all the required stations and reconfigure them such that they functionally fit within the floor space.

The configurations module 1003 also determines the zone configuration and floor area for the end-to-end pill dispensing, packaging, and delivery system.

The data analyzed by the requirements and factors module 1001 and configurations module 1003 is used by the restrictions module 1005 to further analyze the space layout in connection with the user's requirements and factors.

The constraints received are divided into two sections, general layout constraints and user defined constraints. General layout constraints include zones or floor area constraints, space or height constraints, blocked space that cannot be used, or constraints due to other fixtures.

There may also be user defined constraints. These may include user preferences on station locations, user preferences on layout, user preferences on additional displays or manual stations, or height constraints based on height of their operators of other constraints due to liability.

Since both types of constraints are crucial for framing the layout, they are considered and applied by the restrictions module 1005.

The layout generator 1007 receives all the inputs and analyses performed by the requirements and factors module 1001, configurations module 1003, and restrictions module 1005. These inputs may be in a specific computer language code, such as algorithmic mathematical language code. It takes into account all the requirements, factors, station configurations, zone configurations, and any general or user defined restrictions to produce a layout. The layout produced includes location of every station. The station locations are placed in systematic order such that the system may perform in the most efficient manner.

At block 1009, the layout generated by the layout generator 1007 is optimized using algorithms and specific functions, the optimization results in refining the process flow and resource utilization in most efficient manner. A final layout is produced at 1011.

The outcome may also be a resource allocation layout that is customized and suitable for the specific location. This layout would convey the number of pill dispensing robots and one or more of the stations discussed above, such as printer station, sealing station etc. The optimized layout assists the facility that will house the system in obtaining the resources described in the layout to have an efficient system that fits their space and provides productivity.

It will be apparent to those skilled in the art that various modifications and variations may be made in the disclosed embodiments of the disclosed methods and systems without departing from the spirit or scope of the invention. Thus, it is intended that the present invention covers the modifications and variations of the embodiments disclosed above provided that the modifications and variations come within the scope of any claims and their equivalents.

What is claimed is:

1. A computerized automated method for managing end-to-end medical pill dispensing and packaging process in a private computerized networked environment, wherein the medical pills are being packaged in a blister pack for a patient in accordance with a prescription that is specific to the patient, wherein the private computerized networked environment includes one or more computing devices comprising processor hardware, the method including the steps of:
using a central server, activating a prescription extraction flow module to extract a prescription from a client computer, wherein the prescription includes patient data and medication data, wherein the patient data includes the patient's name and patient's date of birth, and the medication data includes a list of one or more medical pills prescribed for the patient, the quantity of pills for each of the one or more medical pills prescribed for the patient, and the hours of administration (HOA) of the one or more medical pills, wherein the central server includes a processor, a system memory, and a decision logic module, wherein the processor is coupled to the system memory through an input/output (I/O) interface, and the decision logic module is communicatively connected to the processor, wherein the prescription is extracted based on instructions provided by the processor;
storing the extracted prescription in the system memory of the central server;
processing the stored prescription to separate the patient data from the medication data;
developing a pack process flow plan, wherein the pack processing flow plan is developed as a result of communications between the decision logic module and a pack processing flow module, wherein the processing flow plan includes step by step sequential flow for dispensing and packaging the one or more medical pills prescribed for the patient into a blister pack;
in accordance with the pack processing flow plan, the processor executing instruction thereby resulting in a mobile robot car retrieving an empty blister pack, wherein the mobile robot car includes a plurality of wheels and a receiver for receiving path guidance instructions from the processor;
in accordance with the pack processing flow plan, the decision logic module selecting a pill dispenser for populating the retrieved empty blister pack with the one or more medical pills prescribed for the patient, wherein the selected pill dispenser is within the computerized networked environment and communicatively coupled to the central server through a private network;
the mobile robot car transporting the retrieved empty blister pack to the selected pill dispenser, wherein the transporting instructions are executed by the processor and provided to the mobile robot;
the processor sending the medication data from the stored prescription to the selected pill dispenser and instructing the pill dispenser to dispense pills into the empty blister pack in accordance with the hours of administration of the prescription, wherein such instructions resulting in the dispensing and populating of the blister pack;
automatically formatting the extracted prescription in a label printable format based on instructions executed by the processor;
in accordance with the pack processing flow plan, selecting a hardware printing device and the processor providing instructions to the hardware printing device thereby resulting in the printing of a label that is to be placed on the blister pack;
the processor directing the mobile robot car to transport the populated blister pack to the hardware printing device, wherein once reaching the hardware printing device, the printed label is placed on the populated blister pack;
in accordance with the pack processing flow plan, the processor executing instruction thereby resulting in the mobile robot car transporting the populated blister pack from the hardware printing device to a sealing station where the label is sealed onto the blister pack;
in accordance with the pack processing flow plan, the processor executing instruction thereby resulting in the mobile robot car transporting the populated blister pack from the sealing station to a storage station for its storage; and
executing a deep learning module thereby to analyze the execution of the pack processing flow plan and revising the pack processing flow plan for its next execution.

2. The computerized automated method of claim 1, wherein using the central server, activating the prescription extraction flow module to extract a prescription from a client computer, further comprises:
authorizing the central server to obtain access to the client computer for extracting the prescription, wherein the prescription is stored in a database of the client computer;
the central server pinging the client computer's database to extract the prescription;
receiving the prescription from the client computer;
saving the prescription to an Rx folder in the memory of the client computer; and
analyzing the prescription to separate patient data from medical data.

3. The computerized automated method of claim 1, wherein developing the pack process flow plan, comprising:
the processor pinging resources that are in communicatively connected through the private computerized networked environment, wherein the resources include one or more robot cars, parking stations, pill dispensers, printer stations, sealing stations, and unloading stations;
determining resource functionality, wherein the determination includes evaluating resource functions errors;
obtaining resource schedule, wherein the schedule includes resource downtime, resource current availability, resource pipeline, and resource open time slots;
analyzing resource functionality and resource schedule to develop an optimal process flow path.

4. The computerized automated method of claim 3, wherein the optimal path is a path that packages the prescription in the blister pack in the shortest amount of time.

5. The computerized automated method of claim 3, wherein the optimal path is the shortest by distance.

6. The computerized automated method of claim 3, wherein the optimal path is the utilization of resources based on their schedule availability.

7. The computerized automated method of claim 1, further comprising scheduling one or more pill dispensers for dispensing pills into the blister pack, the scheduling comprising:
analyzing the prescription to determining the type of medical pills and the quantity of each medical pill required;
querying a first pill dispenser to determine the medical pills available in its storage;
querying a second pill dispenser to determine the medical pills available in its storage;
transporting the blister pack to the first dispenser if a determination is made that the first pill dispenser contains all the type of medical pills and the quantity of each medical pill required to fill the blister pack;
transporting the blister pack to the second pill dispenser if a determination is made that the first pill dispenser does not contain all the type of medical pills and the quantity of each medical pill required to fill the blister pack and that the second pill dispenser contains all type of medical pills and the quantity of each medical pill required to fill the blister pack;
transporting the blister pack to either the first or the second pill dispenser for a partial fill of the prescription and then transporting the partially filled blister pack to the first or the second pill dispenser for the remaining fill of the prescription.

8. The computerized automated method of claim 7, wherein the transporting the blister pack to either the first or the second pill dispenser for a partial fill includes selecting one of the two pill dispensers comprising:
determining percentage of prescription that can be filled by the first pill dispenser based on the medical pills available in its storage;
determining percentage of prescription that can be filled by the second pill dispenser based on the medical pills available in its storage;
selecting the pill dispenser with the higher percentage as the first pill dispenser to partially fill the blister pack.

9. The computerized automated method of claim 1, executing the deep learning module comprises:
analyzing the path taken by the robot car to transport the blister pack from one location to another location to dispense medical pills into the blister pack and package the blister pack;
analyzing the total amount of time taken to perform each step of the pill dispensing and packaging process;
analyzing delays and downtime in performing each step of the pill dispensing and packaging process;
analyzing distance travelled by the robot car in performing each step of the pill dispensing and packaging process; and
scoring the results of the path taken, total time, delays and downtime, and distance travelled.

10. The computerized automated method of claim 9, further comprising:
developing one or more alternative paths for dispensing and packaging the blister pack; and
scoring each of the one or more alternative paths based on their total time, delays and downtime, and distance travelled and scoring the alternative paths.

11. The computerized automated method of claim 10, wherein revising the pack processing flow plan includes selecting the path with the highest score.

12. The computerized automated method of claim 11, wherein the deep learning module continuously revises the pack processing flow plan after filling of each prescription.

13. A computerized automated method for managing medical pill inventory in an end-to-end medical pill dispensing and medical pill packaging system, wherein the medical pills are dispensed by a pill dispenser and packaged in a blister pack for a patient in accordance with a prescription that is specific to the patient, wherein the end-to-end medical pill dispensing and medical pill packaging system includes one or more computing devices that include one or more processors, the computerized automated method for managing medical pill inventory comprising:
using a central server to retrieve a plurality of prescriptions from a client computer's database, wherein the central server includes a processor, a system memory, and a decision logic module, wherein the processor is coupled to the system memory through an input/output (I/O) interface, and the decision logic module is communicatively connected to the processor, wherein the prescription is extracted based on instructions provided by the processor, wherein the prescription includes a list of one or more medical pills prescribed for the patient, the quantity of pills for each of the one or more medical pills prescribed for the patient, and the hours of administration (HOA) of the one or more medical pills,
storing the plurality of retrieved prescription in the system memory of the central server;
processing the plurality of prescriptions to determine the total type and number of pills required to fill the plurality of prescriptions;
querying each pill dispenser that is communicatively connected to the central server through a private network to determine the number and type of pills available in each pill dispenser's pill storage compartments;
determine if the number and type of pills available in each pill dispenser's pill storage are sufficient to dispense and fill the plurality of retrieved prescriptions; and
ordering medical pills if the pills in the dispenser's pill storage are less than the pills required to fill the plurality of prescriptions.

14. The computerized automated method for managing medical pill inventory of claim 13, wherein the plurality of prescriptions are prescriptions that are to be filled within a certain time period.

15. The computerized automated method for managing medical pill inventory of claim 14, wherein the time period is a single day.

16. The computerized automated method for managing medical pill inventory of claim 14, wherein the time period is one week.

17. The computerized automated method for managing medical pill inventory of claim 13, further comprising:
using a central server, retrieving a second set of plurality of prescriptions from the client computer's database, wherein the second set of plurality of prescriptions, wherein the second set of plurality of prescriptions are to be filled at a future time;
processing the second set of plurality of prescriptions to determine the total type and number of pills required to fill the plurality of prescriptions;
querying each pill dispenser to determine number and type of pills available in each pill dispenser's pill storage compartments;
determine if number and type of pills available in each pill dispenser's pill storage are sufficient to dispense and fill the plurality of retrieved prescriptions and the second set of plurality of prescriptions; and
ordering medical pills if the pills in the dispenser's pill storage are less than the pills required to fill both the plurality of prescriptions and the second set of plurality of prescriptions.

18. The computerized automated method for managing medical pill inventory of claim 17, further comprising providing the cost of ordering the pills required to fill the second set of plurality of prescriptions.

19. The computerized automated method for managing medical pill inventory of claim 17, further comprising providing a budgeting tool having a plurality of adjustable controls, wherein adjusting each control provides a cost of building pill inventory.

20. A computerized automated method for providing an optimized space layout for housing an end-to-end medical pill dispensing, medical pill packaging, and medical pill inventory system, wherein the medical pills are dispensed by a pill dispenser and packaged in a blister pack for a patient in accordance with a prescription that is specific to the patient, wherein the end-to-end medical pill dispensing and medical pill packaging system resides in a computerized networked environment and includes one or more computing devices that include one or more processors, the computerized automated method for providing an optimized space layout comprising:
receive a plurality of requirements and factors, wherein the requirements and factors include number of blister packs to be processed and typical volume of blister pack processing to be expected by the location;
receive a plurality of configurations, wherein the configurations include a dimensional layout of the space available to house the end-to-end medical pill dispensing, medical pill packaging, and medical pill inventory system receive a plurality of restrictions, wherein the restrictions include blocked spaces where equipment cannot be placed, blocked zones where equipment cannot be placed, height restrictions, and temporary or permanent fixtures;
analyze the requirements, factors, configurations, and restrictions to develop a layout for housing the end-to-end medical pill dispensing, medical pill packaging, and medical pill inventory system;
evaluating and optimizing the developed layout; wherein the optimization includes refining the layout based on logical and sequential flow of dispensing and packaging a blister pack; and
producing a final layout.
